(19) United States
(12) Patent Application Publication
(10) Pub. No.: US 2008/0026379 A1
(43) Pub. Date: Jan. 31, 2008

(54) Nucleotide analogs

(76) Inventors: Suhaib M. Siddiqi, Burlington, MA (US); Edyta Krzymanska-Olejnik, Brookline, MA (US); Herman Antonio Orgueira, Cambridge, MA (US); Xiaopeng Bai, Providence, RI (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/496,262

(22) Filed: Jul. 31, 2006

(51) Int. Cl. C12Q 1/68; C07H 21/00

(52) U.S. Cl. 435/6; 536/25.32

(57) ABSTRACT

The invention provides nucleotide analogs for use in sequencing nucleic acid molecules.

### FIELD OF THE INVENTION

The invention relates to nucleotide analogs and methods for sequencing a nucleic acid using the nucleotide analogs.

### BACKGROUND

New sequencing technologies, based on single-molecule measurements, have been proposed. These proposals include sequencing strategies based on the observation of an interaction of particular proteins with DNA, or by using ultra high resolution scanned probe microscopy. See, e.g., Rigler, et al., J. Biotechnol., 86(3):161 (2001); Goodwin, P. M., et al., Nucleosides & Nucleotides, 16(5-6):543-550 (1997); Howorka, S., et al., Nature Biotechnol., 19(7):636-639 (2001); Meller, A., et al., Proc. Nat'l. Acad. Sci., 97(3):1079-1084 (2000); Driscoll, R. J., Nature, 346(6281):294-296 (1990).

Sequencing-by-synthesis methodology that results in sequence determination, but without consecutive base incorporation, has also been proposed. See, Braslavsky, et al., Proc. Nat'l Acad. Sci., 100: 3960-3964 (2003). Bulky fluorophores that impede sequential base incorporation can be an impediment to base-over-base sequencing. Even when the label is removed, some fluorescently-labeled nucleotides hinder subsequent base incorporation possibly due to the residue of the linker that is left behind after label removal.

A need therefore exists for nucleotide analogs that promote accurate base-over-base incorporation in sequencing-by-synthesis reactions, resulting in greater read lengths.

### SUMMARY OF THE INVENTION

The present invention provides nucleotide analogs and methods of using nucleotide analogs in sequencing. A nucleotide analog of the invention comprises a removable detectable moiety that is attached to a nucleotide analog, and that upon removal of the detectable moiety, does not substantially hinder subsequent nucleotide (or nucleotide analog) incorporation. Before removal of a detectable moiety, analogs of the invention may allow only limited base addition in any given cycle of template-dependent nucleotide incorporation.

Nucleotide analogs of the present invention include those depicted by Formula I:

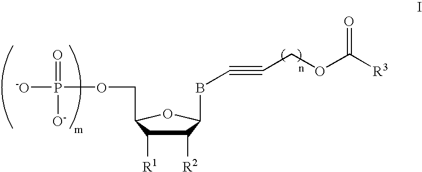

wherein,

B is selected from the group consisting of a purine, a pyrimidine, and analogs thereof,

R¹ is selected from the group consisting of OH and an —O-blocking group,

R² is selected from the group consisting of H and OH,

R³ is selected from the group consisting of

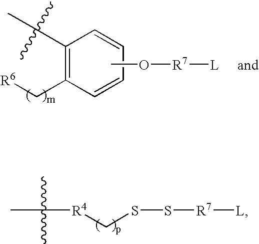

R⁴ is selected from the group consisting of O, S and NR⁵,

R⁵ is selected from the group consisting of H and alkyl,

R⁶ is N₃,

R⁷ is an aliphatic moiety,

L is a label,

m, at each occurrence, independently is an integer from 1 to 3; n, at each occurrence, independently is an integer from 1 to 18; and p, at each occurrence, independently is an integer from 1 to 11.

B may selected from the group consisting of cytosine, uracil, thymine, adenine, guanine, and analogs thereof, such as for example, inosine.

In certain embodiments, R⁴ is O. In other embodiments, R⁶ is NH₂.

In certain embodiments, n is 1. In other embodiments, m is 1.

L may be an optically detectable label, such as a fluorescent label. An optically detectable label may be selected from the group consisting of cyanine, rhodamine, fluoroscein, coumarin, BODIPY, alexa and conjugated multi-dyes. In some embodiments, the optically detectable label is Cy3 or Cy5.

In some embodiments, R¹ is OH or a phosphate moiety.

The disclosure also provides for a nucleic acid polymer comprising a nucleotide analog, wherein the nucleotide analog is represented by Formula II:

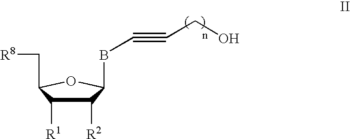

wherein,

B is selected from the group consisting of a purine, a pyrimidine, and analogs thereof,

R¹ is selected from the group consisting of OH and an —O-blocking group,

R² is selected from the group consisting of H and OH,

R⁸ is a phosphodiester linkage connecting the nucleotide analog to a sugar of an adjacent nucleotide in the nucleic acid, and

n, at each occurrence, independently is an integer from 1 to 18.

In general, methods of sequencing a nucleic acid template provided herein comprise exposing a nucleic acid template hybridized to a primer having a 3′ end to a polymerase which catalyzes nucleotide additions to the primer complementary to the template or extended primer, and to plural nucleotide analogs disclosed herein under conditions to permit the polymerase to add the nucleotide analog to the primer, or extended primer, detecting the nucleotide analog added to the primer, removing the label from the nucleotide analog, and repeating these steps thereby to determine the sequence of the template. The method steps may be repeated at least three times, or, in some embodiments, six times, ten times, more than fifteen or higher times or more than 25 times.

In preferred embodiments, the nucleic acid template is immobilized to a solid support. In other embodiments, the templates immobilized in an array at a density sufficient to detect and sequence single molecules individually.

The label may be removed from the nucleotide analogs by, for example, exposure to a reducing agent such as dithiothreitol, tris(2-carboxyethyl)phosphine and tris(2-chloropropyl)phosphate.

While the invention is exemplified herein with fluorescent labels, the invention is not so limited and can be practiced using nucleotides labeled with any detectable label, preferably an optically detectable label, such as chemiluminescent labels, luminescent labels, phosphorescent labels, fluorescence polarization labels, as well as charge labels.

A detailed description of the certain embodiments of the invention is provided below. Other embodiments of the invention are apparent upon review of the detailed description that follows.

### BRIEF DESCRIPTION OF THE DRAWINGS

FIG. 1 depicts a synthetic route A to an intermediate compound that may be used to prepare nucleotide analog disclosed herein having a label attached to a base. Route B depicts a synthetic route for an intermediate used in route A.

FIG. 2 depicts a synthetic route to a nucleotide analog disclosed herein.

FIG. 3 depicts a reaction of a nucleotide analog and a reducing agent.

FIG. 4 depicts a synthetic route to a nucleotide analog disclosed herein and removal of a label from a nucleotide analog.

### DETAILED DESCRIPTION OF THE INVENTION

The invention relates generally to nucleotide analogs that, when used in sequencing reactions, allow extended base-over-base incorporation into a primer in a template-dependent sequencing reaction. Nucleotide analogs of the invention include nucleotide triphosphates having a linker between the base portion of the nucleotide and a detectable label, wherein the linker is cleavable to produce an un-labeled residue that closely resembles the native (i.e., unlabeled) nucleotide. Such a residue or analog results from contacting a labeled analog with a reducing agent resulting in an un-labeled analog that differs from a native nucleotide only by an alkynyl hydroxyl stub that is out of the plane of the nucleotide polymer helix. Such an analog permits polymerase to recognize the analog as a nucleotide and add bases, and does not affect subsequent base pairing. Analogs of the invention are thus useful in sequencing-by-synthesis reactions in which consecutive bases are added to a primer in a template-dependent manner.

### Nucleotide Analogs

Nucleotide analogs of the invention have the generalized structure:

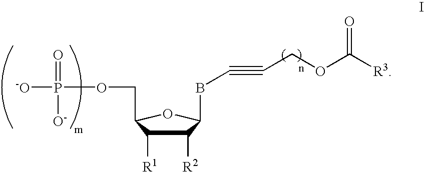

The base B can be, for example, a purine or a pyrimidine. For example, B can be an adenine, cytosine, guanine, thymine, uracil, or hypoxanthine. The base B also can be, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil(pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. Bases useful according to the invention may permit a nucleotide, that includes the base, to be incorporated into a polynucleotide chain by a polymerase and may form base pairs with a base on an antiparallel nucleic acid strand. The term base pair encompasses not only the standard AT, AU or GC base pairs, but also base pairs formed between nucleotides and/or nucleotide analogs comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the nucleotide analog inosine and adenine, cytosine or uracil, where the two hydrogen bonds are formed.

Label L may be any moiety that can be attached to or associated with an oligonucleotide and that functions to provide a detectable signal, and/or to interact with a second label to modify the detectable signal provided by the first or second label, e.g. fluorescence resonance energy transfer (FRET). The label preferably is an optically-detectable label. In one embodiment, the label is an optically-detectable label such as a fluorescent, chemiluminescence, or electrochemically luminescent label. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels. Any appropriate detectable label can be used according to the invention, and numerous other labels are known to those skilled in the art.

The nucleotide analogs of the present invention also can include a moiety R¹ at the 3′ position of the nucleotide sugar that may prevent further extension of the primer after the nucleotide analog has been added to the primer. R¹ thus can include OH, and a —O-blocking agent, such as phosphate, ester, ether, phosphoryl, and the like. Therefore, in one embodiment, the R¹ moiety may be phosphate group rather than a standard hydroxyl group. A phosphoryl may in general be represented by the formula:

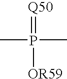

wherein Q50 represents S or O, and R59 represents hydrogen, a lower alkyl or an aryl. When used to substitute, e.g., an alkyl, the phosphoryl group of the phosphorylalkyl may be represented by the general formulas:

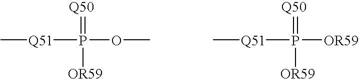

wherein Q50 and R59, each independently, are defined above, and Q51 represents O, S or N. When Q50 is S, the phosphoryl moiety is a “phosphorothioate”.

Alkyl moieties include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” also includes halosubstituted alkyls. Moreover, the term “alkyl” (or “lower alkyl”) includes “substituted alkyls”, which refers to alkyl moieties having substituents replacing a hydrogen on one or more carbons of the hydrocarbon backbone.

In order to prevent or reduce degradation of the primer containing the nucleotide analog or degradation of the nucleotide analogs, the nucleotide analog can further comprise a non-bridging sulfur on the α phosphate group of the nucleotide.

R² may be selected from H and OH. R³ is selected from the group consisting of

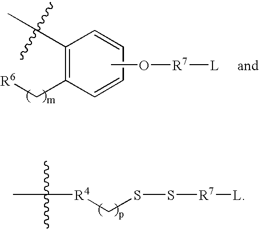

R⁴ may be selected from the group consisting of O, S and NR₅. In some embodiments, R⁴ is O.

R⁵ may be selected from the group consisting of H and alkyl; R⁶ may be selected from the group consisting of N₃ and NR⁵. R⁷ may be any acceptable chemical linker that is capable of associated or bonding the label to a molecular chain that includes R³. For example, R⁷ may be an aliphatic moiety, such as a linear, branched, cyclic alkane, alkene, or alkyne. In certain embodiments, aliphatic groups may be linear or branched and have from 1 to about 20 carbon atoms. In some embodiments, R⁶ is N₃.

The integer m, at each occurrence, independently is an integer from 1 to 3; n, at each occurrence, independently is an integer from 1 to 18, and p, at each occurrence, independently is an integer from 0 to 11. In some embodiments, n is 1. In other embodiments, m is 1.

### Nucleic Acid Sequencing

The invention also includes methods for nucleic acid sequence determination using the nucleotide analogs described herein. The nucleotide analogs of the present invention are particularly suitable for use in single molecule sequencing techniques. Such techniques are described for example in U.S. patent application Ser. No. 10/831,214 filed April 2004; Ser. No. 10/852,028 filed May 24, 2004; Ser. No. 10/866,388 filed Jun. 10, 2005; Ser. No. 10/099,459 filed Mar. 12, 2002; and U.S. Published Application 2003/013880 published Jul. 24, 2003, the teachings of which are incorporated herein in their entireties. In general, methods for nucleic acid sequence determination comprise exposing a target nucleic acid (also referred to herein as template nucleic acid or template) to a primer that is complementary to at least a portion of the target nucleic acid, under conditions suitable for hybridizing the primer to the target nucleic acid, forming a template/primer duplex.

Target nucleic acids include deoxyribonucleic acid (DNA) and/or ribonucleic acid (RNA). Target nucleic acid molecules can be obtained from any cellular material obtained from an animal, plant, bacterium, virus, fungus, or any other cellular organism, or may be synthetic DNA. Target nucleic acids may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid molecules may also be isolated from cultured cells, such as a primary cell culture or a cell line. The cells from which target nucleic acids are obtained can be infected with a virus or other intracellular pathogen. Nucleic acid molecules may also include those of animal (including human), wild type or engineered prokaryotic or eukaryotic cells, viruses or completely or partially synthetic RNAs or DNAs. A sample can also be total RNA extracted from a biological specimen, a cDNA library, or genomic DNA.

Nucleic acid typically is fragmented to produce suitable fragments for analysis. In one embodiment, nucleic acid from a biological sample is fragmented by sonication. Test samples can be obtained as described in U.S. Patent Application 2002/0190663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, target nucleic acid molecules can be from about 5 bases to about 20 kb, about 30 kb, or even about 40 kb or more. Nucleic acid molecules may be single-stranded, double-stranded, or double-stranded with single-stranded regions (for example, stem- and loop-structures).

Single molecule sequencing includes a template nucleic acid molecule/primer duplex that is immobilized on a surface such that the duplex and/or the nucleotides (or nucleotide analogs) added to the immobilized primer are individually optically resolvable. The primer, template and/or nucleotide analogs are detectably labeled such that the position of an individual duplex molecule is individually optically resolvable. Either the primer or the template is immobilized to a solid support. The primer and template can be hybridized to each other and optionally covalently cross-linked prior to or after attachment of either the template or the primer to the solid support.

In general, methods for facilitating the incorporation of a nucleotide analog as an extension of a primer include exposing a target nucleic acid/primer duplex to one or more nucleotide analogs disclosed herein and a polymerase under conditions suitable to extend the primer in a template dependent manner. Generally, the primer is sufficiently complementary to at least a portion of the target nucleic acid to hybridize to the target nucleic acid and allow template-dependent nucleotide polymerization. The primer extension process can be repeated to identify additional nucleotide analogs in the template. The sequence of the template is determined by compiling the detected nucleotides, thereby determining the complementary sequence of the target nucleic acid molecule.

Any polymerase and/or polymerizing enzyme may be employed. A preferred polymerase is Klenow with reduced exonuclease activity. Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al., 1998, Proc Natl Acad. Sci. USA 95:14250->5).

Other DNA polymerases include, but are not limited to, ThermoSequenase®, 9°Nm™, Therminator®, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347(1975)).

Unincorporated nucleotide analog molecules may be removed prior to or after detecting. Unincorporated nucleotide analog molecules may be removed by washing.

A template/primer duplex is treated to remove the label and/or to cleave the molecular chain attaching the label to the nucleotide. The steps of exposing template/primer duplex to one or more nucleotide analogs and polymerase, detecting incorporated nucleotides, and then treating to (1) remove the label, (2) remove the label and at least a portion of the molecular chain associating the label to the nucleotide or (3) cleave the molecular chain. These steps can be repeated, thereby identifying additional bases in the template nucleic acid, the identified bases can be compiled, thereby determining the sequence of the target nucleic acid. In some embodiments, at least some portions of the remaining molecular chain and/or label are not removed, for example, in the last round of primer extension.

In some embodiments, a nucleotide analog, after removal of the label and portions of the molecular chain connecting the label to the nucleotide can be represented by:

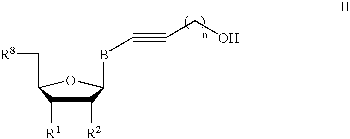

wherein,

B can be any base, and can be for example selected from the group consisting of a purine, a pyrimidine, and analogs thereof. R¹ can be selected from the group consisting of OH and phosphoryl. In some embodiments, R¹ is a phosphate group. R² may be selected from the group consisting of H and OH. R⁸ can be a phosphodiester linkage connecting the nucleotide analog to a sugar of an adjacent nucleotide in the nucleic acid, or a phosphoryl group. The integer n, at each occurrence may be independently an integer from 1 to 18.

One embodiment of a method for sequencing a nucleic acid template includes exposing a nucleic acid template to a primer capable of hybridizing to the template to a polymerase capable of catalyzing nucleotide addition to the primer and a labeled nucleotide analog disclosed herein under conditions to permit the polymerase to add the nucleotide analog to the primer. A method for sequencing may further include identifying or detecting the incorporated labeled nucleotide. A cleavable bond may then be cleaved, removing at least the label from the nucleotide analog. The exposing, detecting, and removing steps are repeated at least once. In certain embodiments, the exposing, detecting, and removing steps are repeated at least three, five, ten or even more times. The sequence of the template can be determined based upon the order of incorporation of the labeled nucleotides.

In another embodiment, a method for sequencing a nucleic acid template includes exposing a nucleic acid template to a primer capable of hybridizing to the template and a polymerase capable of catalyzing nucleotide addition to the primer. The polymerase is, for example, Klenow with reduced exonuclease activity. The polymerase adds a labeled nucleotide analog disclosed herein. The method may include identifying the incorporated labeled nucleotide. Once the labeled nucleotide is identified, the label and at least a portion of a molecular chain connecting the label to the nucleotide analog are removed and the remaining portion of the molecular chain includes a free hydroxyl group. The exposing, incorporating, identifying, and removing steps are repeated at least once, preferably multiple times. The sequence of the template is determined based upon the order of incorporation of the labeled nucleotides.

Removal of a label from a disclosed labeled nucleotide analog and/or cleavage of the molecular chain linking a disclosed nucleotide to a label may include contacting or exposing the labeled nucleotide with a reducing agent. Such reducing agents include, for example, dithiothreitol (DTT), tris(2-carboxyethyl)phosphine (TCEP), tris(3-hydroxy-propyl)phosphine, tris(2-chloropropyl)phosphate (TCPP), 2-mercaptoethanol, 2-mercaptoethylamine, cystein and ethylmaleimide. Such contacting or exposing the reducing agent to a labeled nucleotide analog may occur at a range of pH, for example at a pH of about 5 to about 10, or about 7 to about 9.

The above-described methods for sequencing a nucleic acid template can further include a step of capping a molecular chain, for example, after the label has been removed.

After addition of the nucleotide analog to the primer, any optional 3′ phosphate moiety can be removed enzymatically. In one embodiment, an optional phosphate can be removed using alkaline phosphatase or T₄ polynucleotide kinase. Suitable enzymes for removing optional phosphate include, any phosphatase, for example, alkaline phosphatase such as shrimp alkaline phosphatase, bacterial alkaline phosphatase, or calf intestinal alkaline phosphatase.

Reference to the following figures illustrating exemplary reaction schemes and nucleotide analogs are intended in no way to limit the scope of this invention but are provided to illustrate how to prepare and use the compounds of the present invention. Many other embodiments of this invention will be apparent to one skilled in the art.

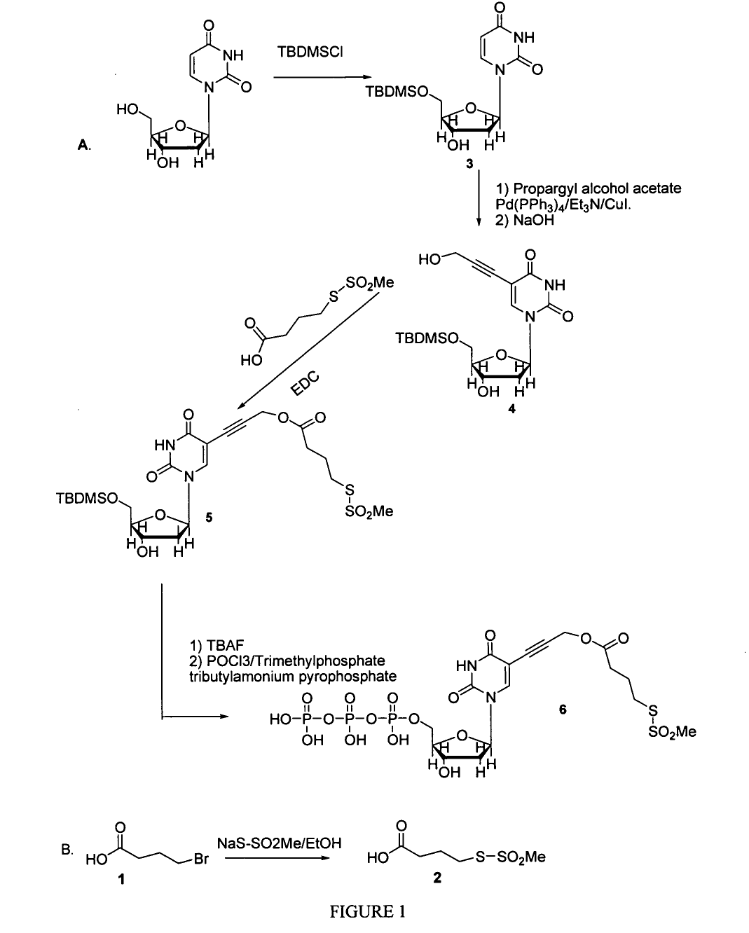

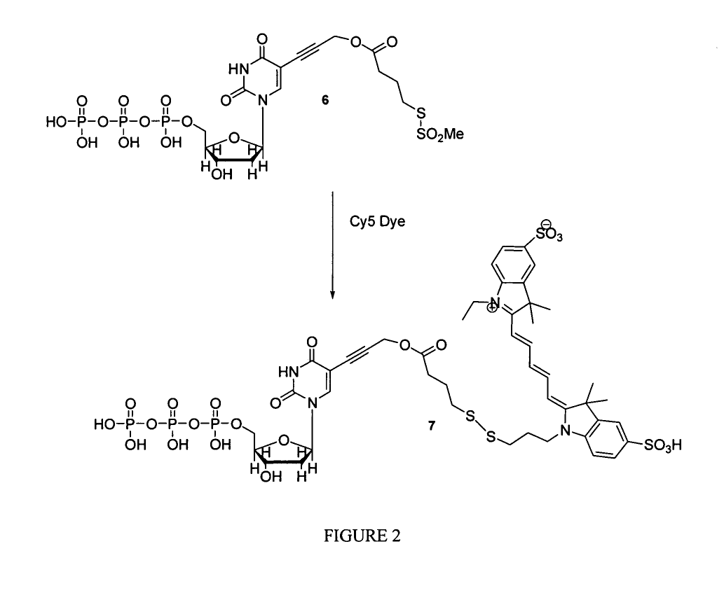

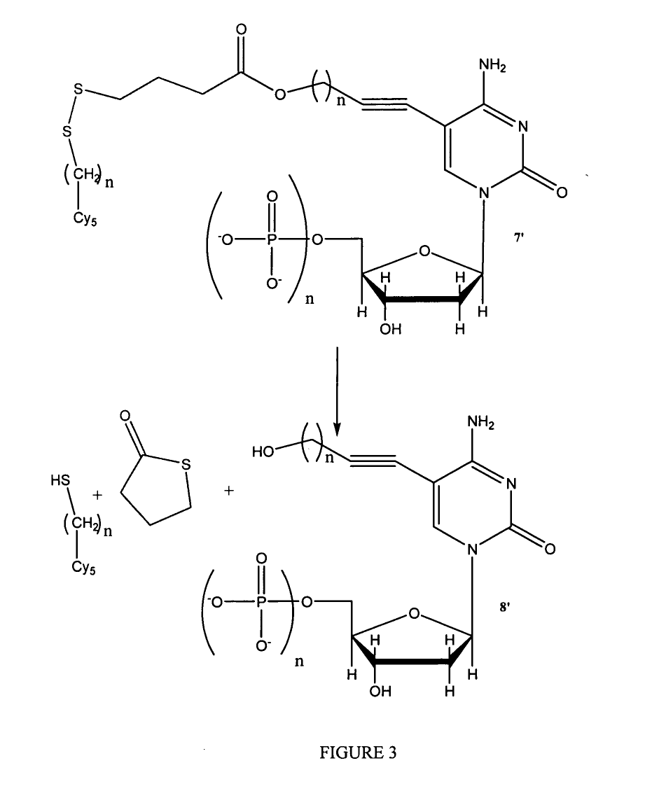

FIGS. 1 and 2 depict an exemplary synthetic route to an exemplary labeled nucleotide analog of this disclosure. Compound 2 is used as a precursor reagent to synthesize the labeled nucleotide analog 7. FIG. 3 indicates that upon exposure to a reducing agent, the label from 7′ is removed as 10 and a substantial portion of moiety linking the label to the nucleotide has also been removed by the formation of the heterocyclic compound 9. The reaction conditions upon exposure to a reducing agent may include a pH of about 7.4 and a temperature of 37° C. The nucleotide analog 8 includes only a short alkynyl moiety remaining after removal of the label.

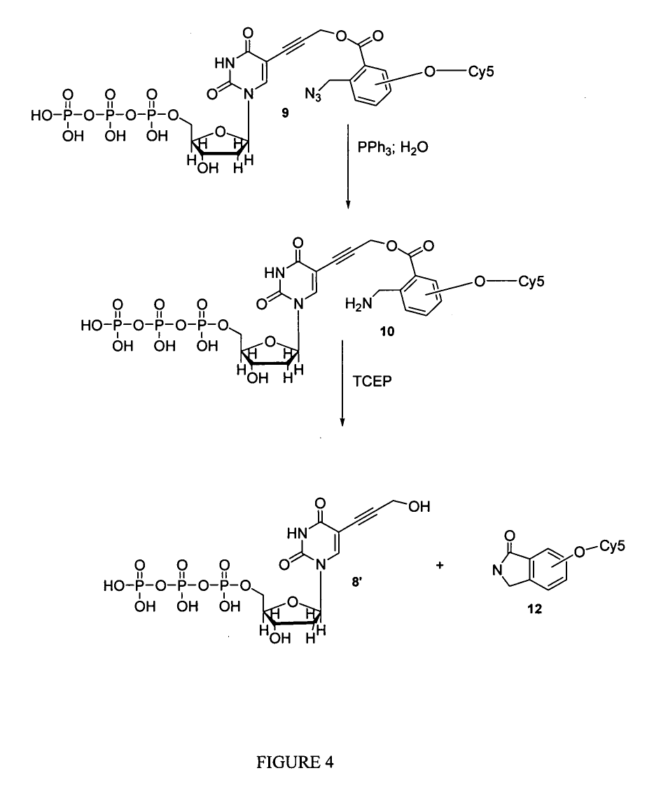

FIG. 4 depicts an exemplary partial synthetic route to an exemplary labeled nucleotide analog of this disclosure. The azide of compound 9 is converted to an amine using triphenylphosphine, resulting in the labeled nucleotide analog 10. Upon exposure to TCEP, the label of 10 is removed as cyclic compound 12; resulting in nucleotide analog 8′ that includes only an alkynyl stub.

### Detection

Any detection method may be used to identify an incorporated nucleotide analog that is suitable for the type of label employed. Thus, exemplary detection methods include radioactive detection, optical absorbance detection, e.g., UV-visible absorbance detection, optical emission detection, e.g., fluorescence or chemiluminescence. Single-molecule fluorescence can be made using a conventional microscope equipped with total internal reflection (TIR) objective. The detectable moiety associated with the extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T.G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached target nucleic acids.

The present invention provides for detection of molecules from a single nucleotide to a single target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode can be used. Full field imaging with a two-stage image intensified CCD camera also can be used. Additionally, low noise cooled CCD can also be used to detect single fluorescent molecules.

The detection system for the signal may depend upon the labeling moiety used. For optical signals, a combination of an optical fiber or charged couple device (CCD) can be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems can be used. Various physical orientations for the detection system are available and discussion of important design parameters is provided in the art.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached target nucleic acid target molecule/primer complex in the presence of a polymerase. Total internal reflectance fluorescence microscopy is then used to visualize the attached target nucleic acid target molecule/primer complex and/or the incorporated nucleotides with single molecule resolution.

Fluorescence resonance energy transfer (FRET) can be used as a detection scheme. FRET in the context of sequencing is described generally in Braslavasky, et al., Proc. Nat'l Acad. Sci., 100: 3960-3964 (2003), incorporated by reference herein. In an embodiment, a donor fluorophore is attached to the primer, polymerase, or template. Nucleotides added for incorporation into the primer comprise an acceptor fluorophore that is activated by the donor when the two are in proximity.

Measured signals can be analyzed manually or preferably by appropriate computer methods to tabulate results. Preferably, the signals of millions of analogs are read in parallel and then deconvoluted to ascertain a sequence. The substrates and reaction conditions can include appropriate controls for verifying the integrity of hybridization and extension conditions, and for providing standard curves for quantification, if desired. For example, a control nucleic acid can be added to the sample. The absence of the expected extension product is an indication that there is a defect with the sample or assay components requiring correction.

### EXAMPLE

The 7249 nucleotide genome of the bacteriophage M13 mp18 is sequenced using nucleotide analogs of the invention.

Purified, single-stranded viral M13 mp18 genomic DNA is obtained from New England Biolabs. Approximately 25 ug of M13 DNA is digested to an average fragment size of 40 bp with 0.1 U Dnase I (New England Biolabs) for 10 minutes at 37° C. Digested DNA fragment sizes are estimated by running an aliquot of the digestion mixture on a precast denaturing (TBE-Urea) 10% polyacrylamide gel (Novagen) and staining with SYBR Gold (Invitrogen/Molecular Probes). The DNase I-digested genomic DNA is filtered through a YM10 ultrafiltration spin column (Millipore) to remove small digestion products less than about 30 nt. Approximately 20 pmol of the filtered DNase I digest was then polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62.). The average dA tail length is about 50+/−5 nucleotides. Terminal transferase is then used to label the fragments with Cy3-dUTP. Fragments are then terminated with dideoxyTTP (also added using terminal transferase). The resulting fragments are again filtered with a YM10 ultrafiltration spin column to remove free nucleotides and stored in ddH2O at −20° C.

Epoxide-coated glass slides are prepared for oligo attachment. Epoxide-functionalized 40 mm diameter #1.5 glass cover slips (slides) are obtained from Erie Scientific (Salem, N.H.). The slides are preconditioned by soaking in 3×SSC for 15 minutes at 37° C. Next, a 500 pM aliquot of 5′ aminated polydT(50) (polythymidine of 50 bp in length with a 5′ terminal amine) is incubated with each slide for 30 minutes at room temperature in a volume of 80 ml. The resulting slides have poly(dT50) primer attached by direct amine linker to the epoxide. The slides are then treated with phosphate (1 M) for 4 hours at room temperature in order to passivate the surface. Slides are then stored in polymerase rinse buffer (20 mM Tris, 100 mM NaCl, 0.001% Triton® X-100 (polyoxyethylene octyl phenyl ether), pH 8.0) until used for sequencing.

For sequencing, the slides are placed in a modified FCS2 flow cell (Bioptechs, Butler, Pa.) using a 50 um thick gasket. The flow cell is placed on a movable stage that is part of a high-efficiency fluorescence imaging system built around a Nikon TE-2000 inverted microscope equipped with a total internal reflection (TIR) objective. The slide is then rinsed with HEPES buffer with 100 mM NaCl and equilibrated to a temperature of 50° C. An aliquot of the M13 template fragments described above is diluted in 3×SSC to a final concentration of 1.2 nM. A 100 ul aliquot is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains M13 template/oligo(dT) primer duplex. The temperature of the flow cell is then reduced to 37° C. for sequencing and the objective is brought into contact with the flow cell.

For sequencing, cytosine triphosphate analog, guanidine triphosphate analog, adenine triphosphate analog, and uracil triphosphate analog, each having a fluorescent label, such as a Cy5, attached to the base via a molecular chain, such as the labeled nucleotide analogs disclosed herein. The analogs are stored separately in buffer containing 20 mM Tris-HCl, pH 8.8, 10 mM MgSO₄, 10 mM (NH₄)₂SO₄, 10 mM HCl, and 0.1% Triton® X-100 (polyoxyethylene octyl phenyl ether), and 100 U Klenow exo⁻ polymerase (NEN). Sequencing proceeds as follows.

First, initial imaging is used to determine the positions of duplex on the epoxide surface. The Cy3 label attached to the M13 templates is imaged by excitation using a laser tuned to 532 nm radiation (Verdi V-2 Laser, Coherent, Inc., Santa Clara, Calif.) in order to establish duplex position. For each slide only single fluorescent molecules imaged in this step are counted. Imaging of incorporated nucleotides as described below is accomplished by excitation of a cyanine-5 dye using a 635 nm radiation laser (Coherent). 5 uM of a Cy5-labeled CTP analog as described above is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes)). An oxygen scavenger containing 30% acetonitrile and scavenger buffer (134 ul HEPES/NaCl, 24 ul 100 mM Trolox in MES, pH 6.1, 10 ul DABCO in MES, pH 6.1, 8 ul 2M glucose, 20 ul NaI (50 mM stock in water), and 4 ul glucose oxidase) is next added. The slide is then imaged (500 frames) for 0.2 seconds using an Inova301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 ul) and HEPES/NaCl (60 ul).

Next, the fluorescent label (e.g., the cyanine-5) is removed or cleaved off of the incorporated CTP analogs. The Cy5 label is removed by introduction into the flow cell of 50 mM TCEP for 5 minutes, after which the flow cell was rinsed 5 times each with SSC/HEPES/SDS (60 ul) and HEPES/NaCl (60 ul), and the remaining nucleotide is capped with 50 mM iodoacetamide for 5 minutes followed by rinsing 5 times each with SSC/HEPES/SDS (60 ul) and HEPES/NaCl (60 ul). The scavenger is applied again in the manner described above, and the slide is again imaged to determine the effectiveness of the cleave/cap steps and to identify non-incorporated fluorescent objects.

The procedure described above is then conducted 100 nM Cy5dATP analog, followed by 100 nM Cy5dGTP analog, and finally 500 nM Cy5dUTP, each as described above. The procedure (expose to nucleotide, polymerase, rinse, scavenger, image, rinse, cleave, rinse, cap, rinse, scavenger, final image, removal of optional phosphate group) is repeated exactly as described for ATP, GTP, and UTP except that Cy5dUTP is incubated for 5 minutes instead of 2 minutes. Uridine is used instead of thymidine due to the fact that the Cy5 label is incorporated at the position normally occupied by the methyl group in thymidine triphosphate, thus turning the dTTP into dUTP. In all 64 cycles (C, A, G, U) are conducted as described in this and the preceding paragraph.

Once 64 cycles are completed, the image stack data (i.e., the single molecule sequences obtained from the various surface-bound duplex) is aligned to the M13 reference sequence.

The alignment algorithm matches sequences obtained as described above with the actual M13 linear sequence. Placement of obtained sequence on M13 is based upon the best match between the obtained sequence and a portion of M13 of the same length, taking into consideration 0, 1, or 2 possible errors. All obtained 9-mers with 0 errors (meaning that they exactly matched a 9-mer in the M13 reference sequence) are first aligned with M13. Then 10-, 11-, and 12-mers with 0 or 1 error are aligned. Finally, all 13-mers or greater with 0, 1, or 2 errors are aligned.

All publications, patents, and patent applications cited herein are hereby expressly incorporated by reference in their entirety and for all purposes to the same extent as if each was so individually denoted. The patent applications entitled “Nucleotide Analogs” filed on even date herewith (Attorney Docket Numbes: HEL-040; HEL-039) are each expressly incorporated by reference.

### EQUIVALENTS

While specific embodiments of the subject invention have been discussed, the above specification is illustrative and not restrictive. Many variations of the invention will become apparent to those skilled in the art upon review of this specification. Contemplated equivalents of the nucleotide analogs disclosed here include compounds which otherwise correspond thereto, and which have the same general properties thereof, wherein one or more simple variations of substituents or components are made which do not adversely affect the characteristics of the nucleotide analogs of interest. In general, the components of the nucleotide analogs disclosed herein may be prepared by the methods illustrated in the general reaction schema as described herein or by modifications thereof, using readily available starting materials, reagents, and conventional synthesis procedures. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A labeled nucleotide analog of Formula I: 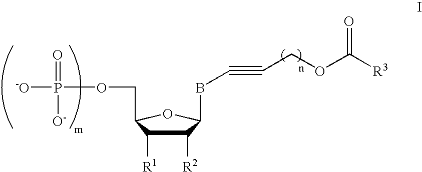
wherein,
B is selected from the group consisting of a purine, a pyrimidine, and analogs thereof,
R¹ is selected from the group consisting of OH and —O-blocking agent,
R² is selected from the group consisting of H and OH,
R³ is selected from the group consisting of 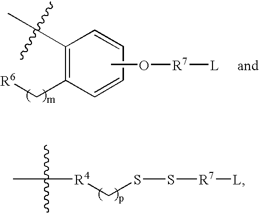
R⁴ is selected from the group consisting of alkyl, O, S, and NR⁵,
R⁵ is selected from the group consisting of H and alkyl,
R⁶ is N₃,
R⁷ is an alkyl linker,
L is a label,
m, at each occurrence, independently is an integer from 1 to 3,
n, at each occurrence, independently is an integer from 1 to 18, and
p, at each occurrence, independently is an integer from 0 to 11.

2. The labeled nucleotide analog of claim 1, wherein B is selected from the group consisting of cytosine, uracil, thymine, adenine, guanine, and analogs thereof.

3. The labeled nucleotide analog of claim 1 or 2, wherein R⁴ is O.

4. The labeled nucleotide analog of claim 1, or 2, wherein R⁴ is selected from the group consisting of CH₂, CHCH₃ or C(CH₃)₂.

5. The labeled nucleotide analog of claim 1, wherein n is 1.

6. The labeled nucleotide analog of claim 1, wherein m is 1.

7. The labeled nucleotide analog of claim 1, where L is an optically detectable label.

8. The labeled nucleotide analog of claim 7, wherein the optically detectable label is a fluorescent label.

9. The labeled nucleotide analog of claim 7, wherein the optically detectable label is selected from the group consisting of cyanine, rhodamine, fluoroscein, coumarin, BODIPY, alexa and conjugated multi-dyes.

10. The labeled nucleotide analog of claim 7, wherein the optically detectable label is Cy3 or Cy5.

11. The labeled nucleotide analog of claim 1, wherein R³ is

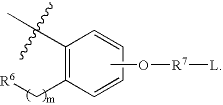

12. The labeled nucleotide analog of claim 11, wherein m is 1.

13. The labeled nucleotide of claim 11, wherein R³ is

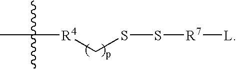

14. The labeled nucleotide of claim 1, wherein R¹ is OH.

15. The labeled nucleotide of claim 1, wherein R⁷ is a C₃-C₇ alkyl.

16. A nucleic acid comprising a nucleotide analog, wherein the nucleotide analog is represented by Formula II: 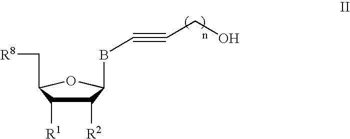
wherein,
B is selected from the group consisting of a purine, a pyrimidine, and analogs thereof,
R¹ is selected from the group consisting of OH and a —O-blocking agent,
R² is selected from the group consisting of H and OH,
R⁸ is a phosphodiester linkage connecting the nucleotide analog to a sugar of an adjacent nucleotide in the nucleic acid, and
n, at each occurrence, independently is an integer from 1 to 18.

17. The nucleic acid analog of claim 16, wherein n is 1, 2 or 3.

18. A method of sequencing a nucleic acid template comprising:
(a) exposing a nucleic acid template hybridized to a primer having a 3′ end to (i) a polymerase which catalyzes nucleotide additions to the primer, and (ii) the nucleotide analog of claim 1 under conditions to permit the polymerase to add the nucleotide analog to the primer;
(b) detecting the nucleotide analog added to the primer in step (a);
(c) removing the label from the nucleotide analog; and
(d) repeating steps (a), (b) and (c) thereby to determine the sequence of the template.

19. The method of claim 18, where step (d) is repeated at least three times.

20. The method of claim 18, wherein the nucleotide analog, after step (c), is represented by Formula II: 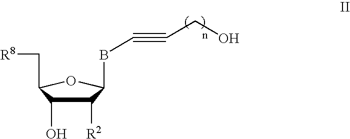
wherein,
B is selected from the group consisting of a purine, a pyrimidine, and analogs thereof,
R² is selected from the group consisting of H and OH,
R⁸ is a phosphodiester linkage connecting the nucleotide analog to the primer, and
n is an integer from 1 to 18.

21. The method of claim 18, wherein during step (a), the template is immobilized to a solid support.

22. The method of claim 18, wherein, during step (c), the label is removed by exposure to a reducing agent.

23. The method of claim 18, wherein the reducing agent is selected from the group consisting of dithiothreitol, tris(2-carboxyethyl)phosphine and tris(2-chloropropyl)phosphate, tris-(3-hydroxypropyl)phosphine, tributylphosphine and sodium dithionate.

24. The method of claim 21, wherein the template are immobilized in an array at a density sufficient to detect and sequence single molecules individually.
